(19) United States
(12) Patent Application Publication
(10) Pub. No.: US 2008/0057578 A1
(43) Pub. Date: Mar. 6, 2008

(54) PROCESS AND SUBSTRATE FOR CULTURING CARTILAGE CELL, MATERIAL FOR REPRODUCING BIOLOGICAL TISSUE CONTAINING CARTILAGE CELL, AND CARTILAGE CELL

(76) Inventors: Kosuke Kuwabara, Hitachi (JP); Akihiro Miyauchi, Hitachi (JP); Toshimasa Uemura, Tsuchiura (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/844,384

(22) Filed: Aug. 24, 2007

(30) Foreign Application Priority Data
Aug. 30, 2006 (JP) 2006-234630

(51) Int. Cl. C12N 5/08

(52) U.S. Cl. 435/366; 435/395

(57) ABSTRACT

There is provided a technology for simple three-dimensional culturing of cartilage cells. The technology includes a method for culturing cartilage cells which comprises seeding cartilage cells 1 on a plurality of projected portions 21 of a culturing substrate 2 having a culture surface on which the projected portions 21 having an equivalent diameter and an interval which are smaller than the equivalent diameter of the cartilage cells 1 to be cultured, placing the culture substrate 2 having the cartilage cells 1 in a culture vessel, and culturing the cartilage cells to form aggregates of cartilage cells.

### BACKGROUND OF THE INVENTION

The present invention relates to a technology for culturing cartilage cells using a culturing substrate, and particularly, it relates to a technology for culturing cartilage cells in a given phenotype using culturing substrate.

Recently, cell culture technologies employed for medical treatment have progressed, and application to regenerative medical treatment and cell engineering is expected much.

The cartilage tissue has weak self-repairing ability, and, hence, although natural repairing can be expected for small injuries, in the case of injuries of more than a certain degree caused in Osteoarthritis (OA), Rheumatoid Arthritis (RA) or the like, natural repairing cannot be expected. The artificial joint replacement which is one of representative treatments of cartilage tissue has limited application since the artificial joints can hardly be used for a long time of more than 10 years because of wear of artificial joints.

Recently, in the field of orthopedics, a technology of culturing and growing in vitro cartilage cells isolated from cartilage taken from patients per se and thereafter transplanting them into cartilage defect has been actively studied for repairing the cartilage defect and has partially been put to practical use. In this case, if planarily cultured cartilage cells are used, sometimes it becomes difficult to dispose the cartilage cells at the desired position or the cartilage cells sometimes dedifferentiate into fibroblasts and do not secrete cartilage matrix (matrix protein) such as collagen type II and aggrecan, and it becomes impossible to construct three-dimensional cartilage tissue.

Generally, when cartilage cells are planarity cultured in a culture vessel having a flat culture surface, such as a laboratory dish (Schale), only a planar sheet can be obtained. In order to dispose the cells at the desired position or secrete the cartilage matrix which is naturally secreted by cells, it is desired to develop three-dimensional culture process. Furthermore, a three-dimensional culture process which constructs a large three-dimensional tissue requires a large-scaled system or needs a high cost.

Therefore, hitherto, spinner culture method has been utilized as one of three-dimensional culture methods. Furthermore, a method of constructing a large cartilage using RWV bioreactor has been utilized as one of the three-dimensional culture methods (see, for example, Patent Document 1).

Patent Document 1: Pamphlet of International Publication 2005/056072A1

However, in the case of using the spinner culture method, mechanical stimulation or damage given to the cells is great, sometimes causing necrosis inside the cells. Therefore, a cell culture method of three-dimensionally constructing the tissue while leaving them at rest is desired.

Moreover, the method of constructing a large cartilage using RWV bioreactor needs the culture medium in a large amount and requires culturing by an expert, and is apt to cause contamination. Thus, it is necessary to carry out the culture in a severe aseptic state with the greatest care.

### SUMMARY OF THE INVENTION

Under the circumstances, the object of the present invention is to provide a method for three-dimensionally and simply culturing cartilage cells. Particularly, the object is to provide a simply utilizable three-dimensional culturing method which cultures spheroidal tissues.

The method for culturing cartilage cells of the present invention is characterized in that cartilage cells to be cultured are seeded on a plurality of projected portions of a culture substrate having a culture surface on which a plurality of the projected portions having an equivalent diameter and an interval which are smaller than the equivalent diameter of the cartilage cells, and the culture substrate having the cartilage cells disposed thereon is placed in a culture vessel, followed by carrying out culturing to form clump of cartilage cells.

According to the present invention, cartilage cells can be three-dimensionally and simply cultured.

Other objects, features and advantages of the invention will become apparent from the following description of the embodiments of the invention taken in conjunction with the accompanying drawings.

### BRIEF DESCRIPTION OF THE DRAWINGS

FIG. 1 is an oblique view showing the cartilage cells and cartilage cell culturing substrate according to one embodiment of the present invention.

FIG. 2 is an oblique view showing the construction of the cartilage cell culturing substrate according to one embodiment of the present invention, and FIG. 2(a) is a general view and FIG. 2(b) is a partially enlarged oblique view of the area A shown in FIG. 2(a).

FIG. 3 shows steps for production of the cartilage cell culturing substrate by nanoimprinting method.

FIG. 4 explains the process of culturing cartilage cells and is a longitudinal sectional view of the cartilage cell culturing kits.

FIG. 5 is a partially enlarged view showing a plurality of projected portions formed on the upper surface of the bottom of the cartilage cell culturing kit produced in the first example.

FIG. 6 shows results of comparison of the amount of glycosaminoglycan secreted by cartilage cells.

FIG. 7 is an electron micrograph of cartilage cells.

FIG. 8 is an electron micrograph of cartilage cells.

FIG. 9 is an electron micrograph of cartilage cells.

FIG. 10 shows spheroid diameter distribution of cartilage cells, and FIG. 10(a) shows the results when the equivalent diameter of the projected portions is 2.0 μm (Example 1) and FIG. 10(b) shows the results when the equivalent diameter of the projected portions is 0.5 μm (Example 2).

FIG. 11 shows photographs for observing the expression of type II collagen in cartilage cells, and in the case of FIG. 11(a), the equivalent diameter of the projected portions is 2.0 μm (Example 1) and in the case of FIG. 11(b), the equivalent diameter of the projected portions is 0.5 μm (Example 2).

### DESCRIPTION OF REFERENCE NUMERALS IN THE DRAWINGS

1: Cartilage cell
2: Cartilage cell culturing substrate
3: Material for regeneration of biological tissue containing cartilage cells
4: Substrate material
5: Mold
6: Depressed portions of mold
7: Culture vessel
8: Cartilage cell culturing kit
21: Projected portion
21A: Uppermost face (top portion)
22: Substrate base
22A: Upper surface of substrate base
g: Interval
r: Equivalent diameter

### DESCRIPTION OF THE PREFERRED EMBODIMENTS

Preferred embodiments of the present invention will be explained in detail taken in conjunction with the drawings.

The present invention has been accomplished on the basis of the findings that in carrying out culturing with disposing a culture medium for culturing and cartilage cells in a cartilage cell culturing substrate used for culturing cartilage cells under corresponding culturing conditions, when a cartilage cell culturing substrate on the culturing surface of which are provided a plurality of projected portions having a given shape and given intervals is used, the cartilage cells can be cultured with controlling them to a given phenotype.

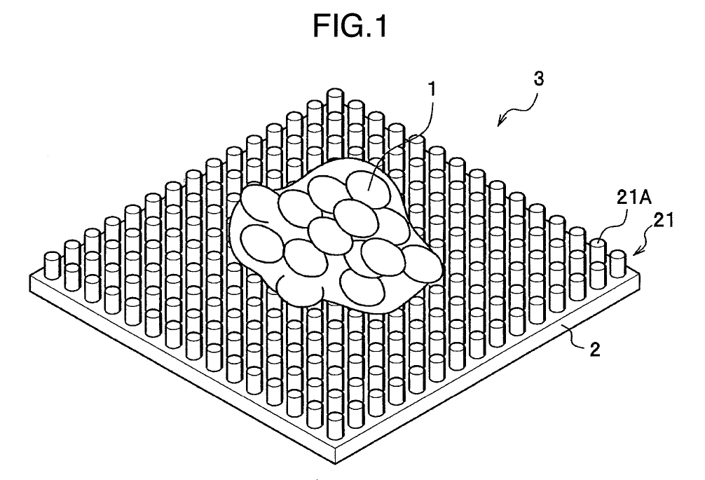

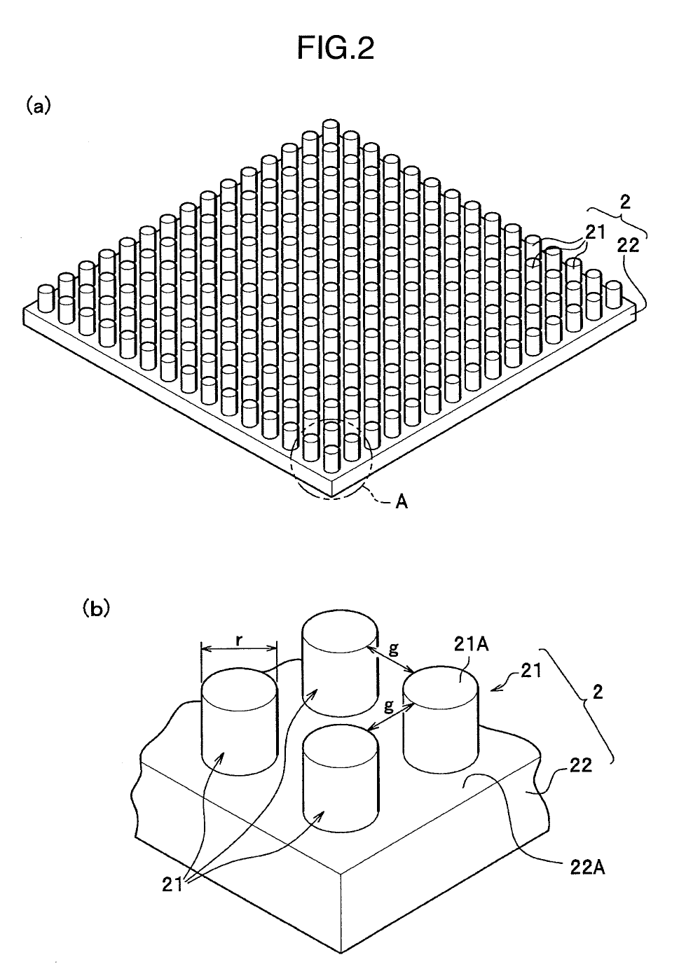

FIG. 1 is an oblique view which shows cartilage cells and a cartilage cell culturing substrate (hereinafter sometimes referred to as “culturing substrate”) of this embodiment. As shown in FIG. 1, the cartilage cells 1 of this embodiment are those which are cultured in contact with uppermost face (top portion) 21A of a plurality of the projected portions 21 having an equivalent diameter smaller than the equivalent diameter of the cartilage cells 1 and formed on the culturing surface. Here, “in contact with uppermost face (top portion) 21A” means that at least a part of the cartilage cells 1 are in such a state that they contact with top portions 21A of a plurality of the projected portions 21 and do not contact with the upper surface 22A of substrate base (FIG. 2(b)). Typically, it is the state where the cartilage cells 1 are adhered extending over the upper side of top portion 21A of a plurality of the projected portions 21.

When the cartilage cells 1 are cultured in this manner, a plurality of cartilage cells 1 such as grown cells and seeded cells agglutinate three-dimensionally to form spheroids (corresponding to the aggregate of cartilage cells). The term “aggregate of cartilage cells” here means the state of the cartilage cells 1 contacting with each other, and includes the states of the cartilage cells 1 sticking to each other, lying one upon another, adhering to each other, and others.

<<Cartilage cells 1>>

Cartilage cells 1 used for culturing are not particularly limited so long as they can be cultured on the cartilage cell culturing substrate 2, and are optionally selected from known cartilage cells 1. For example, in the case of applying to regenerative medical treatment of human, the cartilage cells 1 are preferably those which originate from human, but may be those which originate from non-human animals so long as they are cartilage cells 1 suitable for the diseased part of patients. Furthermore, the cartilage cells 1 may be those which are prepared from cartilage of living bodies, those which are differentiated and derived from stem cells to the cartilage cells 1, or those which are used after subculture.

Especially, it is most suitable to use cartilage cells 1 of individuals per se which are subjected to regenerative medical treatment (particularly preferably, mesenchymal stem cells originating from bone marrow or synovial membrane), because there is no need to carry out immune suppression in transplantation. In case the corresponding cartilage cells 1 cannot be obtained from the individual per se, there can be used cartilage cells 1 originating from the same kind of donor as the individual (especially preferably, mesenchymal stem cells originating from bone marrow or synovial membrane). In this case, it is necessary to carry out selection of donor which causes less rejection and immune suppression in transplantation.

In other words, it is possible to select suitable cells from available cartilage cells 1 depending on the purpose and apply them to this embodiment.

The shape of underside of the cartilage cells 1 is not necessarily circular. Therefore, in this embodiment, when size of the cartilage cells 1 is specified, the expression “diameter” which assumes only the circle is not used, but “equivalent diameter” is used. As for the definition of equivalent diameter of cartilage cells 1, the definition of equivalent diameter of the projected portion 21 mentioned hereinafter can be referred to.

<<Cartilage Cell Culturing Substrate 2>>

FIG. 2 is an oblique view showing the construction of cartilage cell culturing substrate 2 of this embodiment, and FIG. 2(a) is a general view and FIG. 2(b) is a partially enlarged oblique view showing the area A in FIG. 2(a).

As shown in FIG. 2(a), the cartilage cell culturing substrate 2 comprises a substrate base 22 and a plurality of projected portions 21 formed on the culturing surface (for example, upper surface) of the substrate base 22. Spheroids are formed by carrying out the control to three-dimensionally agglutinate the cartilage cells 1 by the projected portions 21 in culturing the cartilage cells 1 on the cartilage cell culturing substrate 2.

<<Projected Portion 21>>

As shown in FIG. 2(b), the projected portions 21 have a given equivalent diameter r of the uppermost face 21A and are arranged at a given interval g.

The shape of the uppermost face 21A is not necessarily circular. Therefore, in this embodiment, when the size of the uppermost face 21A is specified, the expression “diameter” which assumes only the circle is not used, but “equivalent diameter” is used.

Here, “equivalent diameter” is a diameter of the uppermost face 21A of the projected portion 21 or a length equivalent to the diameter, and when the shape of the uppermost face 21A is circular, it is the diameter of the circle and when it is rectangular, it is the length of one side of the rectangle, and when the shape is neither circular nor rectangular, for example, a circle equivalent diameter can be used. The circle equivalent diameter is a diameter specified when the shape of the uppermost face 21A which is not necessarily circular is assumed to be circular. For example, there are an area-circle equivalent diameter which is a diameter of a circle having the same area as of the uppermost face 21A, a perimeter-circle equivalent diameter which is a diameter of a circle having the same perimeter as of the uppermost face 21A, a circumscribed circle equivalent diameter which is a diameter of a circle circumscribed about the shape of the uppermost face 21A, an inscribed circle equivalent diameter which is a diameter of a circle inscribed in the shape of the uppermost face 21A, and the like. These can be optionally selected depending on the shape of the uppermost face 21A.

The equivalent diameter r of projected portions 21 is preferably smaller than the equivalent diameter of cartilage cells 1 in order to reduce the contact area with the cartilage cells 1. Thus, when cartilage cells 1 are cultured on the uppermost face 21A of the projected portions 21, since the contact area of the underside of the cartilage cells 1 with the culture solution increases, and exchanging of nutrient and effete matter between cartilage cells 1 and the culture solution can be accelerated, a desired effect can be exhibited when the cartilage cells 1 form spheroids.

The lower limit of the equivalent diameter r is not particularly limited so long as a plurality of the projected portions 21 can be formed with a uniform working precision.

Specifically, the equivalent diameter r of the projected portions 21 is preferably not less than 10 nm and not more than 10 μm, more preferably not less than 0.2 μm and not more than 5.0 μm.

The form of arrangement of the projected portions 21 can be variously specified so long as the interval g is satisfied. For example, the form of the arrangement of the projected portions 21 is preferably two-dimensional square lattice form or zigzag lattice form for maintaining uniformity of the effect in the same area where the projected portions are formed.

The interval g between the projected portions 21 is specified to be the shortest distance between the outer periphery of the uppermost face 21A of the projected portion 21 and the outer periphery of the uppermost face 21A of the projected portion 21 adjacent to the former projected portion 21. For example, as shown in FIG. 2, in the case of the arrangement of the projected portions 21 being in the form of two-dimensional square lattice, the interval between the projected portions 21 is the length g.

In this embodiment, the interval g between the projected portions 21 is set to be smaller than the equivalent diameter of the cartilage cells 1 to be cultured. By setting in this way, the cartilage cells 1 do not enter into the spaces between the projected portions 21 and are cultured on the uppermost face 21A of the projected portions 21.

The height of the projected portions 21 is set so that a culture solution can be sufficiently provided on the upper surface 22A of the substrate base between the projected portions 21 and a part of the cartilage cells 1 cultured on the uppermost face 21A of the projected portions 21 can be inhibited from contacting with the upper surface 22A of the substrate base. A height of the projected portions 21 of, for example, 0.1 μm or more is enough, and the height is more suitably longer than the equivalent diameter r of the projected portions 21. On the other hand, from the viewpoint of structural strength, it is preferred to set the upper limit of the height of the projected portions 21 to be 100 times the equivalent diameter of the projected portions 21.

Specifically, the height of the projected portions 21 is preferably not less than 10 nm and not more than 1 mm, more preferably not less than 0.1 μm and not more than 100 μm.

The shape of the projected portions 21 in the direction of height is not particularly limited, and may be in the form of column, pyramid or cone, or inverted pyramid or cone, and besides the shape or modification of outer peripheral part is not particularly limited. The shape of the projected portions 21 in the direction of height may be such as tapering from the base to the top or tapering from the base to the top with a thick part in the top portion.

The materials of the cartilage cell culturing substrate 2 comprising projected portions 21 and substrate base 22 are not particularly limited, and are optionally selected depending on the desired working precision, surface characteristics, optical characteristics and strength. Specifically, there may be used thermoplastic resins such as polyethylene, polypropylene, polyvinyl alcohol, polyvinylidene chloride, polyethylene terephthalate, polyvinyl chloride, polystyrene, ABS resin, AS resin, acrylic resin, polyamide, polyacetal, polybutylene terephthalate, glass-reinforced polyethylene terephthalate, polycarbonate, modified polyphenylene ether, polyphenylene sulfide, polyetherether ketone, liquid crystal polymer, fluororesin, polyarate, polysulfone, polyether sulfone, polyamideimide, polyetherimide and thermoplastic polyimide, thermosetting resins such as phenolic resin, melamine resin, urea resin, epoxy resin, unsaturated polyester resin, alkyd resin, silicone resin, diallyl phthalate resin, polyamide bismaleimide and polybisamide triazole, and blends of two or more of them. Moreover, inorganic materials such as quartz and glass can also be used.

The projected portions 21 and the substrate base 22 may be monolithically formed from the same material or may be formed from different materials by adhering the projected portions 21 to the upper surface 22A of the substrate base. It is more preferred to form monolithically the projected portions 21 and the substrate base 22 from the same material because higher strength can be obtained.

Furthermore, the culturing surface of the cartilage cell culturing substrate 2 including the projected portions 21 and the upper surface 22A of the substrate base can be subjected to various treatments according to the purposes.

For example, there may be carried out surface treatments necessary for culturing of cartilage cells 1, such as hydrophilic and hydrophobic treatments by immersion in solvents containing oxidizing agents such as hydrogen peroxide and ozone, irradiation with ultraviolet rays, gas phase treatment such as plasma treatment, coating with protein such as polylysine, albumin, collagen, fibronectin, fibrinogen, vitronectin and laminin by immersion in a solution thereof, metal coating by electroless plating or vapor deposition, coating with a temperature sensitive coating material, and surface modification with light, electron rays or corpuscular beams.

These surface treatments may be carried out over the whole surface or to the limited areas of the cartilage cell culturing substrate 2. For example, different surface treatments may be carried out on a part of the projected portions 21 and the other projected portions 21, or the different surface treatments may be carried out on the projected portions 21 and the other parts of the substrate. Moreover, different surface treatments may be carried out on the uppermost face 21A of the projected portions 21 and the outer peripheral surface of the projected portions 21.

A material 3 for regeneration of biological tissue containing cartilage cells is constituted of the cartilage cell culturing substrate 2 and the cartilage cells 1, and this material may be implanted in the body of patient.

The cartilage cell culturing substrate 2 which constitutes the material 3 for regeneration of biological tissue containing cartilage cells more preferably comprises a biodegradable material (including hydrolysable material).

The materials of the cartilage cell culturing substrate 2 which constitute the material 3 for regeneration of biological tissue containing cartilage cells include, for example, polysaccharides such as alginic acid, cross-linked alginic acid, chitin, chitosan, hyaluronic acid, cross-linked hyaluronic acid, cellulose, starch, cross-linked starch, and derivatives thereof; proteins such as gelatin, cross-linked gelatin, collagen, casein, fibrin, and albumin; polypeptides such as polyaspartic acid, polyglutamic acid and polylysine; synthetic polymer materials such as polyglycolic acid, polylactic acid, poly(E-caprolactone), glycolic acid/lactic acid copolymer, glycolic acid/carbonate copolymer, polydioxanone and cyanoacrylate polymer; and inorganic materials such as hydroxylapatite, tricalcium phosphate and calcium carbonate. Among them, cartilage cell culturing substrates 2 mainly composed of synthetic polymer materials such as polyglycolic acid, polylactic acid, poly(E-caprolactone) and glycolic acid/lactic acid copolymer are superior in rigidity, stability, flexibility, transparency, heat resistance, moist heat resistance, etc.

The method for producing the cartilage cell culturing substrate 2 of this embodiment will be explained referring to the drawings.

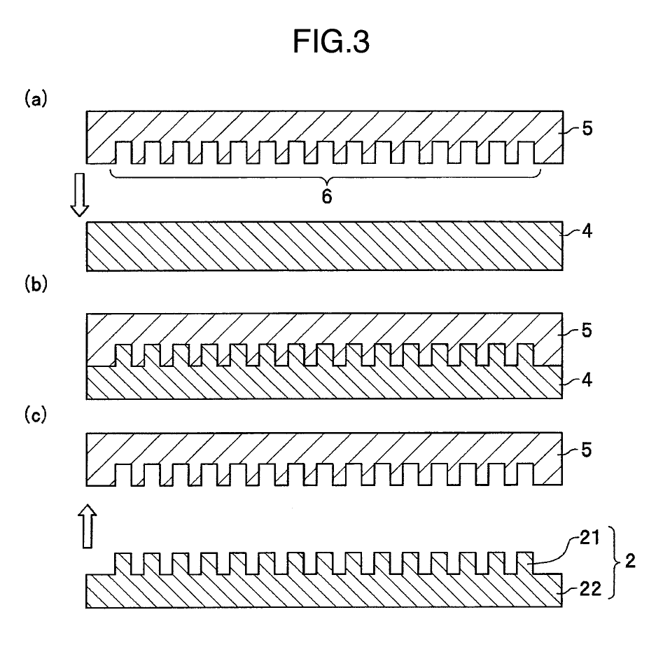

FIG. 3 explains the production steps according to nanoimprinting method which is one example of methods for production of the cartilage cell culturing substrate 2.

First, as shown in FIG. 3(a), a substrate material 4 which is a material for cartilage cell culturing substrate 2 and a mold 5 are prepared. The substrate material 4 is the above-mentioned material suitable for cartilage cell culturing substrate 2. The material of the mold 5 is properly selected from metals such as nickel, inorganic materials such as carbon and silicon, organic materials such as PDMS, resin compositions, etc. depending on the material of the substrate material 4 or working precision of the projected portions 21. The method for forming the mold depressed portions 6 on the surface of the mold 5 is properly selected from cutting method, photolithographic method, electron beam direct drawing method, corpuscular beam method, fine working method such as scanning probe method, self organization of fine particles, or nanoimprinting method using a master made by these methods, molding methods such as casting method and injection molding method, plating method, and the like.

The height, equivalent diameter r or interval g of the projected portions 21 can be controlled by adjusting the depth and size of the depressed portions 6 of the mold 5 and materials of the cartilage cell culturing substrate 2. Furthermore, positions of the projected portions 21 can be controlled by controlling the positions of the mold depressed portions 6.

Next, as shown in FIG. 3(b), at least the surface of the substrate material 4 is softened and the mold 5 having the depressed portions 6 is pressed to the surface of the substrate material 4 to transfer the configuration of the mold depressed portions 6 to the substrate material 4.

Then, as shown in FIG. 3(c), the mold 5 is removed to obtain a cartilage cell culturing substrate 2 comprising the substrate base having projected portions 21 formed monolithically on the upper surface 22A.

In removing the mold 5 from the heated substrate material 4, it is preferred to coat the surface of the mold 5 with a releasing agent such as fluorine-based or silicone-based agent in order to inhibit damage of the upper surface 22A of the substrate base and the projected portions 21.

If necessary, the surface of the cartilage cell culturing substrate 2 having the projected portions 21 can be subjected to surface modification by a method of forming a new layer, such as dipping method, spin coating method, vapor deposition method, plasma polymerization method, ink jet method, or screen printing method, or by heating, light irradiation, electron ray irradiation, plasma treatment, immersion method, or the like. The surface modification treatment may be carried out not only after forming the projected portions 21, and, for example, the surface of the substrate material 4 or mold depressed portions 6 before formation of the projected portions 21 is previously subjected to the surface treatment and the surface of the cell culturing substrate 2 having the projected portions 21 may be subjected to modification treatment simultaneously with the formation of projected portions 21.

The method for producing cartilage cell culturing substrate 2 is not limited to the above nanoimprinting method, and may be optionally selected from cutting method, printing method, ion beam writing method, electron beam direct writing method, laser direct writing method, photolithographic method, casting method, injection molding method, etc., depending on the material of substrate material 4 or working precision of projected portion 21. In the case of using casting method or injection molding method, the mold 5 formed by the above methods can be used.

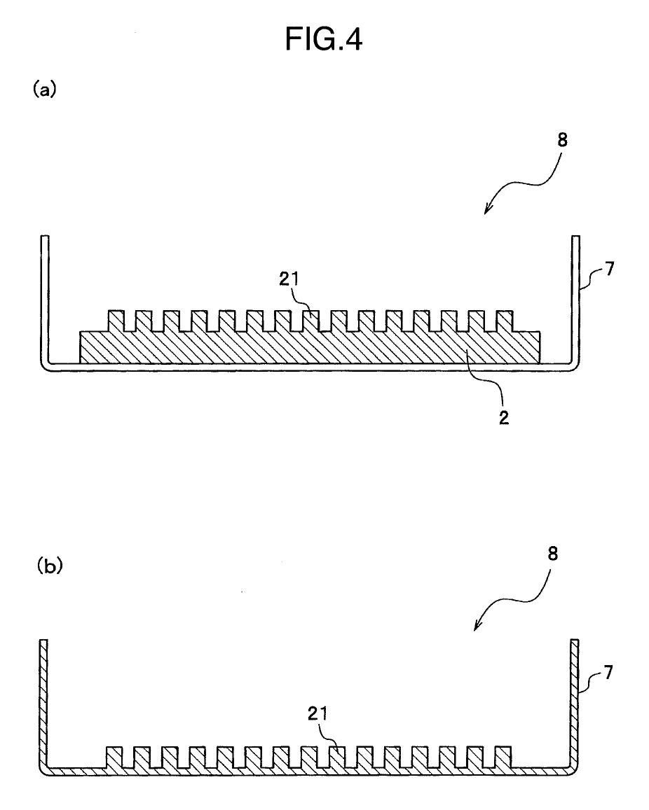

A cartilage cell culturing kit 8 constructed by combining the above cartilage cell culturing substrate 2 and a culture vessel 7 (FIG. 4(a)) and a cartilage cell culturing kit 8 monolithically constructed from them (FIG. 4(b)) are also included in the scope of the present invention. The cartilage cell culturing kit 8 may include a culture solution.

<Method for Culturing Cartilage Cells>

Next, a method for culturing cartilage cells 1 according to this embodiment will be explained referring to the drawings.

As the conditions for culturing cartilage cells 1, there may be optionally applied known culturing conditions selected for cartilage cells 1. For one skilled in the art, the culturing conditions for cartilage cells 1 can be easily selected and culture can be performed under the selected culturing conditions.

The culture medium used may be of known composition suitable for culture of cartilage cells 1, and, for example, there may be used a culture medium for culturing of cartilage cells which is provided by makers. In this case, there may be added a reagent which assists adhesion of cartilage cells 1 to cartilage cell culturing substrate 2 or formation of spheroids.

However, in this embodiment, explanation will be made on the premise that culture is carried out using a culture solution not particularly containing a substance which induces or represses the phenotype of cartilage cells 1 as a culture medium during culturing excluding fixation because it is necessary to clearly explain the controlling effect of the cartilage cell culturing substrate 2 on phenotype of cartilage cells 1.

The incubator used for culture of cartilage cells 1 is not particularly limited so long as the cartilage cells 1 can be cultured, and, for example, a CO₂ incubator which is the same as used for culturing of general cells. Usually, CO₂ incubators are set at a CO₂ concentration of 5%, a temperature of 37° C., and a relative humidity of 80%.

The culturing process of cartilage cells 1 will be explained referring to FIG. 4(a). FIG. 4(a) is a longitudinal sectional view showing one example of cartilage cell culturing kits for culturing cartilage cells 1 and it explains the process of culturing of cartilage cells 1.

First, the cartilage cell culturing substrate 2 is placed on the upper surface of the bottom of the culture vessel 7 in the form of Schale to construct a cartilage cell culturing kit 8. The cartilage cells 1 are seeded on the cartilage cell culturing substrate 2 in the culture vessel 7 together with a culture solution (not shown). In this case, the culturing surface of the cartilage cell culturing substrate 2 is immersed in the culture solution, and the cartilage cells 1 in the culture solution settle down on the cartilage cell culturing substrate 2.

The culture solution and the cartilage cell culturing substrate 2 on which cartilage cells 1 are seeded are left to stand for a given period in a CO₂ incubator.

At this stage, cartilage cells 1 are fixed on (adhere to) the cartilage cell culturing substrate 2 and cultured, and after the fixation, the culture medium may be changed at a given interval. The culture medium used for culturing may be a serum culturemedium, a serum-free culture medium or a culture medium containing supplement or cytokine, and in the case of serum-free culturemedium, it is suitable to change the culture medium at intervals of one day or two days.

After lapse of the given period of time mentioned above, the cartilage sells 1 are used. This given period is not particularly limited and can be extended or shortened according to the desired phenotype of cartilage cells 1 (for example, size of spheroid, the number of cartilage cells 1 contained in the spheroid, the amount of extracellular matrix produced).

In using the cartilage cells 1, only the cartilage cells 1 may be used by peeling off the cartilage cells 1 from the projected portions 21 of the cartilage cell culturing substrate 2, or they may be used as a material 3 for regenerating biological tissue containing cartilage cells which comprises cartilage cells 1 and cartilage cell culturing substrate 2.

For peeling off the cartilage cells 1 which form spheroids on the cartilage cell culturing substrate 2, any methods may be used so long as the cartilage cells 1 can be peeled off from the cartilage cell culturing substrate 2, and known methods can be utilized. For example, a physical method of cutting or drawing the spheroids or a chemical method of using a specific reagent or enzyme can be employed.

FIG. 4(b) is a longitudinal sectional view showing another example of cartilage cell culturing kit 8 for culturing cartilage cells 1. FIG. 4(b) shows a cartilage cell culturing kit 8 comprising a culture vessel 7 and a cartilage cell culturing substrate 2 which are monolithically formed, in which a plurality of the projected portions 21 are directly formed at the culture vessel 7 per se. In the culturing method of cartilage cells 1 of this embodiment, the cartilage cell culturing kit 8 of such construction can also be utilized.

The present invention is not limited to the above embodiment, and various modifications can be made within the scope of the invention.

The cartilage cells 1 and cartilage cell culturing substrate 2 of the present invention will be suitably employed for regeneration of cartilage tissue. For example, it can be expected to utilize suitably the cartilage cells 1 of this embodiment for treatment of deforming articular disease or articular rheumatism.

It can also be expected to carry out the treatment by transplanting the cartilage cells of the present invention in animals having diseases caused in cartilage. The animals in this case are not particularly limited so long as cartilage cells transplantable in them are available, and include various animals such as human, domestic animals (pig, bovine, horse, etc.), mammals for pets (dog, cat, etc.), amphibian, birds, etc.

Therefore, the scope of the present invention is extendable to treating method for patients having troubles with cartilage by transplanting the cartilage cells to the diseased parts of patients.

Furthermore, in this embodiment, the phenotype of cartilage cells 1 is controlled by forming a plurality of projected portions 21 on the cartilage cell culturing substrate 2, but the phenotype of cartilage cells 1 can also be controlled by forming a plurality of depressed portions or forming a rugged structure comprising combination of projected portions 21 and depressed portions.

Moreover, in this embodiment, the culturing surface means an upper surface of the cartilage cell culturing substrate 2 formed as a single-layer sheet, but the shape of the cartilage cell culturing substrate 2 and the position of the culturing surface in the cartilage cell culturing substrate 2 are not limited to those of this embodiment.

For example, the shape of the cartilage cell culturing substrate 2 may be polyhedron, cylinder, laminate of single-layer sheets, etc., and side surface, under surface, inner surface, etc. of the cartilage cell culturing substrate 2 may be used as the culturing surface.

### EXAMPLE

More specific embodiments of the present invention will be explained by the following examples.

### The First Example

### Preparation of Cartilage Cell Culturing Kit 8

The first example is an example of producing a cartilage cell culturing kit 8 in which a cartilage cell culturing substrate 2 having a plurality of projected portions 21 and a culture vessel 7 were monolithically formed (FIG. 4(b)).

The cartilage cell culturing kit 8 of the first example was produced by forming a plurality of projected portions 21 in the circular area of 25 mm in diameter of the upper surface of the bottom of culture vessel 7 in the form of Schale mainly composed of polystyrene and having a thickness of 2 mm. The polystyrene had a molecular weight of 2000-3,840,000. The upper limit of the molecular weight can be extended to 6,000,000.

The projected portions 21 were formed by thermal nanoimprinting method. A mold 5 having ruggedness (FIG. 3) was heated to 130° C. and pressed onto the culture vessel 7 under a pressing pressure of 4 MPa. The mold 5 was a circular silicon wafer having a crystal direction (100) and a diameter of 25 mm. The mold 5 was cooled to 100° C. and then vertically pulled up to form the projected portions 21.

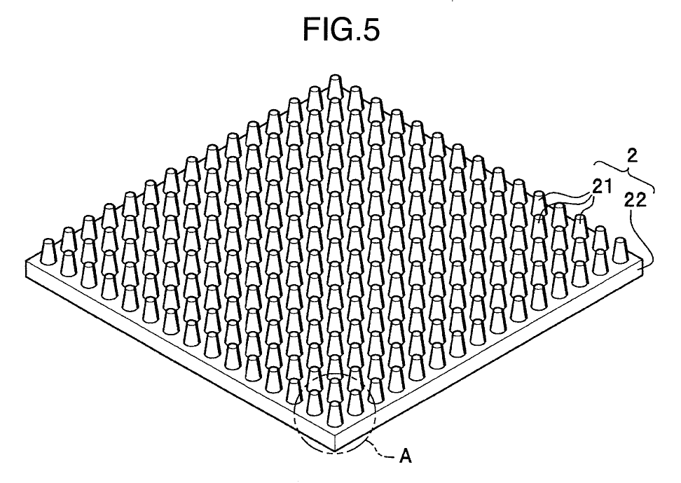

FIG. 5 is a partial oblique view which shows a plurality of projected portions 21 formed on the upper surface of the bottom of the cartilage cell culturing kit 8 produced in the first example.

A plurality of the projected portions 21 were arranged in a planar tetragonal arrangement. Each projected portion 21 was nearly columnar, had an equivalent diameter r of the uppermost face 21A of 400 nm, was widened downwardly, and had an equivalent diameter of the lowermost face of 500 nm. The height was 1 μm, and it can be seen that the ratio of the height and the side was 2 and thus greater than 1. The interval g between a plurality of the projected portions 21 was 1 μm, which was smaller than the equivalent diameter r of the cartilage cells 1 to be cultured.

The projected portions 21 of the thus produced cartilage cell culturing kit 8 hardly peel off from the cartilage cell culturing kit 8 with satisfying the desired shape and interval for control of phenotype of cartilage cells 1.

### The Second Example

### Culturing of Bovine Articular Cartilage Cells

In the second example, bovine articular cartilage cells were cultured by the cartilage cell culturing kit 8 produced by the same method as in the first example, and the resulting cultured cartilage cells (cartilage cell clump) 1 were examined.

<<Culturing of Cartilage Cells 1>>

First, for preparing cartilage cells 1, a cartilage portion of bovine articulation was sliced at about 1 mm square, followed by washing with PBS. Then, the slice was put in a collagenase solution (0.2% collagenase DMEM solution containing amphotericin B (5 ml/500 ml medium)), followed by stirring and shaking at 37° C. for 12-20 hours to decompose collagen. Cartilage cells were obtained by centrifugation (4° C., 1200 rpm, 5 minutes). Then, they were diluted by cell banker so as to obtain 3×10⁷ cells/ml, and were cryopreserved.

Next, for culturing cartilage cells 1, freeze-thawed cartilage cells 1 were seeded on the culturing surface of three kinds of cartilage cell culturing kits 8 (Example 1, Example 2 and Comparative Example mentioned below) so as to give 2×10⁶ cells/cm², respectively. After initial culturing for 3 hours, thereto was added a culture medium (DMEM culture medium (manufactured by Sigma Co., Ltd.) containing 10⁻⁷ M dexamethasone (manufactured by Sigma Co., Ltd.), 10 ng/ml TGF-β3 (manufactured by Sigma Co., Ltd.), 50 μg/ml ascorbic acid (manufactured by Wako Pure Chemical Industries), ITS+Premix (manufactured by BD Co., Ltd.), 40 μg/ml L-proline (manufactured by Sigma Co., Ltd.), and Antibiotic-Antimycotic (manufactured by GIBCO BRL Co., Ltd.), followed by continuing the culturing at 37° C. in 5% CO₂.

Next, explanation will be made on characteristics of the projected portions 21 of the three cartilage cell culturing kits 8 used in the second example.

Example 1: Equivalent Diameter r of the projected portions: 2.0 μm; interval g: 2.0 μm, height: 1.0 μm

Example 2: Equivalent Diameter r of the projected portions: 0.5 μm; interval g: 0.5 μm, height: 1.0 μm

Comparative Example: No projected portions (only the culture vessel 7)

The area where the projected portions were formed in Example 1 and Example 2 was a square of 10 mm×10 mm. The method for producing these cartilage cell culturing kits 8 was the same as in the first example, and, hence, explanation will not be made thereon.

The following evaluations were conducted on Example 1, Example 2 and Comparative Example.

<<Method of Evaluation of Cartilage Cells>>

1. Quantitative Determination of Glycosaminoglycan (GAG) Content:

The amount of GAG in bovine cartilage cultured in Example 1, Example 2 and Comparative Example was measured after 36 days from starting of the culture. The measurement was conducted by quantitative determination of dye using Blyscan Glycosaminoglycan Assay Kit (Biocolor Ltd.).

2. Observation Under Scanning Electron Microscope and Light Microscope:

In order to carry out observation under scanning electron microscope, the cartilage cells 1 cultured in Example 1, Example 2 and Comparative Example were fixed with PBS containing 2.5% glutaraldehyde at 4° C. for 3 hours and incubated in 0.1% osmium tetroxide (OsO₄) at 4° C. for 2 hours, followed by successively carrying out dehydration treatments for 30 minutes in 50, 60, 70, 80, 90, 95, 100% ethanol, respectively. Thereafter, the cartilage cells were lyophilized in t-butyl alcohol, subjected to platinum coating, and observed under HITACHI S-4500 scanning electron microscope (SEM).

The observation under light microscope was conducted using Olympus CKX-41 type microscope.

3. Detection of Cartilage Marker Protein by Immunofluorescent Technique:

After the culturing for 36 days, the cartilage cells were washed three times with TBS-Ca (Tris-buffered saline containing 1 mM CaCl₂, pH 7.6) and fixed with 4% paraformaldehyde at room temperature for 15 minutes. The cartilage cells were washed with TBS-Ca three times, then subjected to methanol treatment at −20° C. for 30 minutes, and subjected to blocking with TBS-Ca containing 5% skim milk for 2 hours. After washing once with TBS-Ca, the primary antibody (anti-mouse type II collagen antibody manufactured by Daiichi Fine Chemical Co., Ltd.) was diluted to 1/500 with TBS-Ca containing 5% skim milk and left to stand at 4° C. overnight. After washing with TBS-Ca two times, thereto was added a secondary antibody (Jackson Immuno Research, FITC-conjugated Affinity Pure F (ab′)₂ fragment goat anti-mouse IgG (H+ L)) diluted to 1/100 with TBS-Ca, followed by treating at room temperature for 30 minutes, washing with TBS-Ca three times, enclosing with a fluorescence fading inhibitor (Vector Laboratories, VECTASHIELD), and carrying out the observation using Carl Zeiss AkioPlan 2 fluorescence microscope.

<<Results of Evaluation of Cartilage Cells>>

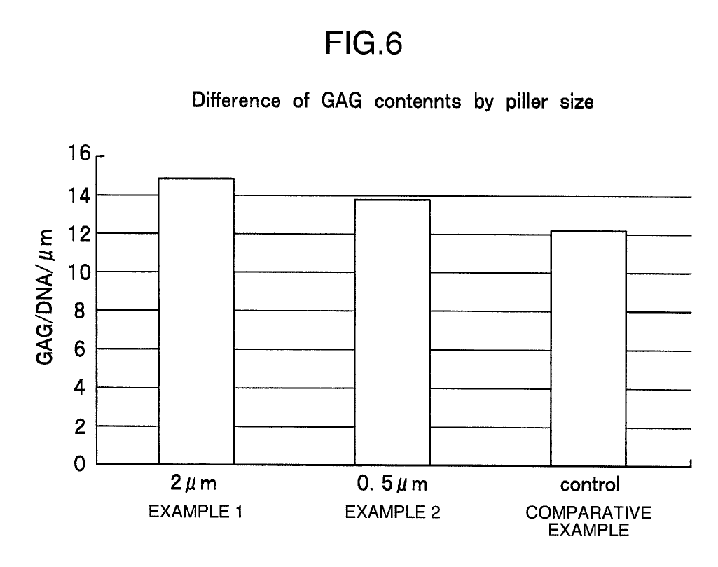

FIG. 6 shows results of comparing the amount of glycosaminoglycan secreted by cartilage cells 1. As shown in FIG. 6, amounts of glycosaminoglycan per unit DNA in Example 1 and Example 2 were larger than in Comparative Example. Furthermore, it can be seen from the results of comparison of Example 1 and Example 2 that the amount of glycosaminoglycan in the cartilage cell culturing kit 8 having projected portions 21 having an equivalent diameter r of 2.0 μm was larger than in the kit having projected portions 21 having an equivalent diameter of 0.5 μm.

It is said that the secreted amount of glycosaminoglycan is particularly larger in cartilage cells 1 as compared with that in other kinds of cells (e.g., fibroblast). That is, it is shown by measurement of amount of glycosaminoglycan that dedifferentiation of cartilage cells 1 occurred in Comparative Example as compared with Example 1 and Example 2. Furthermore, it can be seen from comparison of Example 1 and Example 2 that the degree of differentiation of cartilage cells 1 differs depending on the shape of the projected portions 21.

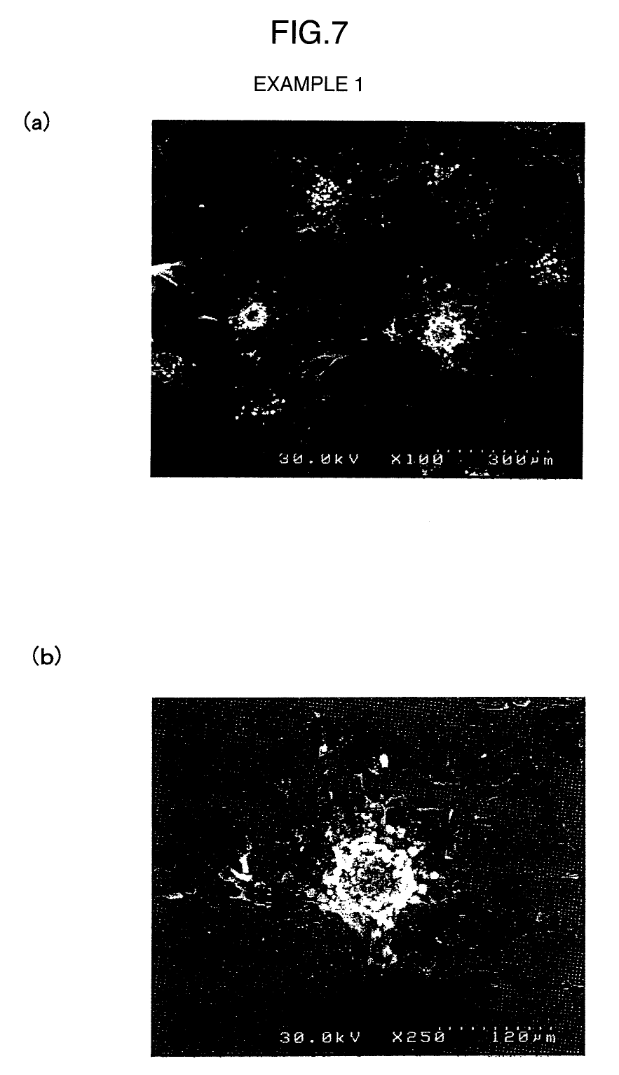

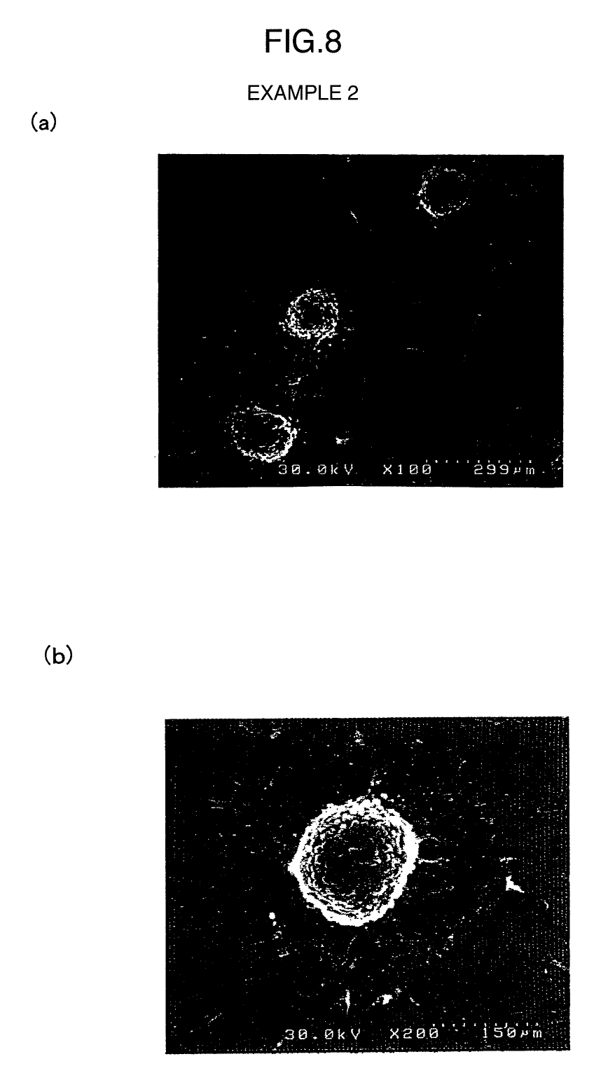

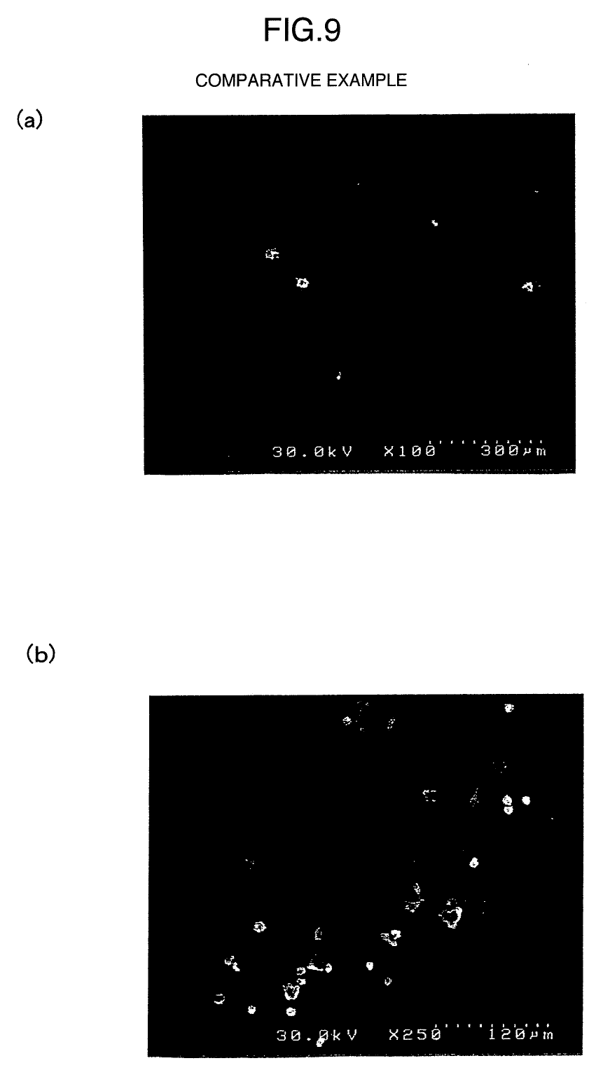

FIG. 7 to FIG. 9 are electron photomicrographs of cartilage cells 1. In FIG. 7 to FIG. 9, (a) and (b) differ in magnification. FIG. 7 and FIG. 8 show that spheroids can readily be formed in Example 1 and Example 2. It is further shown from the comparison of Example 1 and Example 2 that spheroids can be more readily formed in the cartilage cell culturing kit 8 having projected portions 21 having an equivalent diameter r of 0.5 μm than in the kit having projected portions 21 having an equivalent diameter of 2.0 μm. On the other hand, as shown in FIG. 9, cartilage cells 1 formed substantially no spheroids in Comparative Example having no projected portions 2.

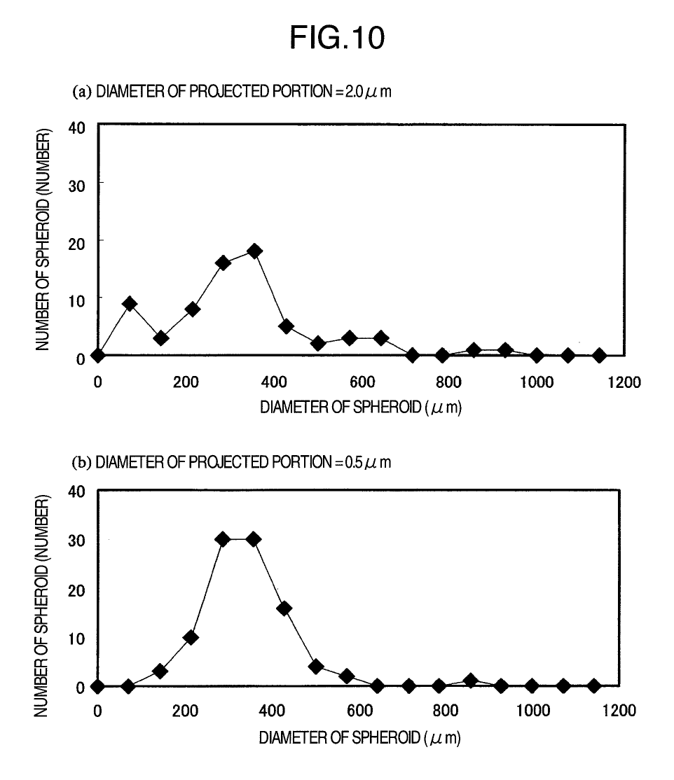

FIG. 10 shows spheroid diameter distribution of cartilage cells 1 which was obtained by the above observation under light microscope, and (a) shows the results when the equivalent diameter r of the projected portions 21 was 2.0 μm (Example 1) and (b) shows the results when the equivalent diameter r of the projected portions 21 was 0.5 μm (Example 2).

FIG. 10 shows the spheroid diameter distribution of cartilage cells 1 on the projected portions 21, and it can be seen that culturing on the projected portions 21 of 0.5 μm in equivalent diameter more satisfactorily controlled the spheroid diameter than on the projected portions 21 of 2.0 μm in equivalent diameter.

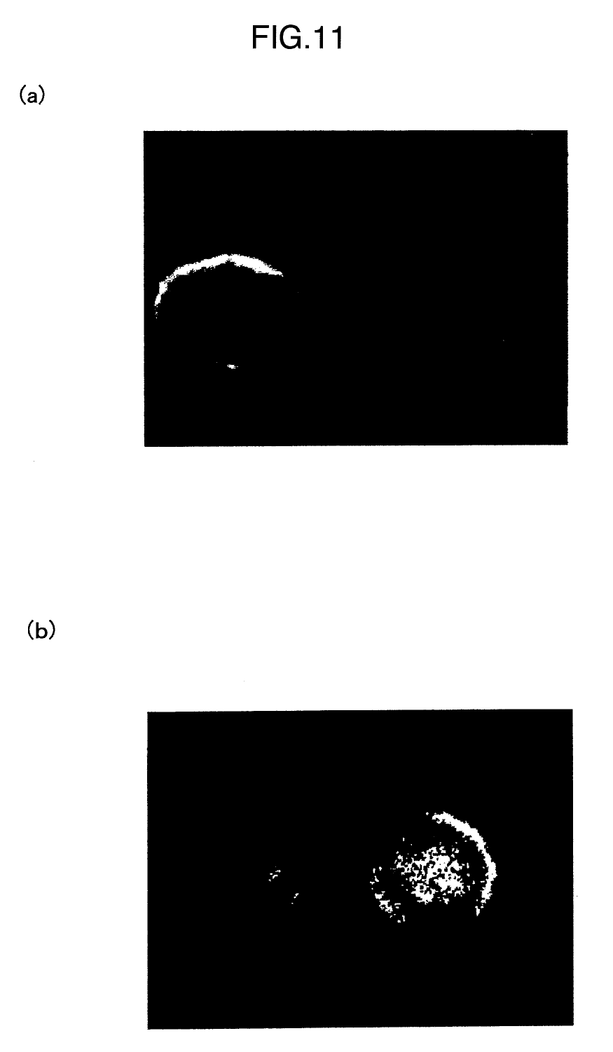

FIG. 11 is for observing development of type II collagen in cartilage cells 1, and in (a), the equivalent diameter r of the projected portions 21 was 2.0 μm (Example 1), and in (b), the equivalent diameter r of the projected portions 21 was 0.5 μm (Example 2). Furthermore, in FIG. 11, collagen type II was developed in the color formed part. It can be seen from FIG. 11 that in both the Example 1 and Example 2 the spheroids formed by culturing on projected portions 21 secreted type II collagen which is a characteristic marker of cartilage. Thus, it is shown that according to the cartilage cell culturing kit of the present invention, dedifferentiation of cartilage cells 1 can be inhibited.

As shown above, the cartilage cell culturing kits 8 having a plurality of projected portions 21 (Example 1 and Example 2) are advantageous for inducing spheroidization of cartilage cells 1 as compared with the cartilage cell culturing kit having no projected portions 21 (Comparative Example).

It should be further understood by those skilled in the art that although the foregoing description has been made on embodiments of the invention, the invention is not limited thereto and various changes and modification may be made without departing from the spirit of the invention and the scope of the appended claims.

1. A method for culturing cartilage cells which comprises seeding cartilage cells on a plurality of projected portions of a culture substrate having a culture surface on which the projected portions having an equivalent diameter and an interval which are smaller than the equivalent diameter of the cartilage cells to be cultured, placing the culture substrate having the cartilage cells disposed thereon in a culture vessel, and culturing the cartilage cells to form aggregates of cartilage cells.

2. A method for culturing cartilage cells according to claim 1, wherein the aggregates of cartilage cells have an equivalent diameter of not less than 10 μm and not more than 5.0 mm.

3. A method for culturing cartilage cells according to claim 1, wherein the cartilage cells constituting the aggregates of cartilage cells contact with each other.

4. A method for culturing cartilage cells according to claim 1, wherein a plurality of the projected portions have an equivalent diameter of not less than 0.01 μm and not more than 10 μm and a height of not less than 0.01 μm and not more than 100 μm.

5. A method for culturing cartilage cells according to claim 1, wherein the cartilage cells are those obtained from living cartilage or those originating from mesenchymal stem cells derived from bone marrow or synovial membrane.

6. A method for culturing cartilage cells according to claim 1, wherein the cartilage cells are those originating from the cells obtained from patients.

7. A method for culturing cartilage cells according to claim 1, wherein the cartilage cells are obtained by culturing using a culture medium containing TGF-β and/or dexamethasone.

8. A method for culturing cartilage cells according to claim 1, wherein the cartilage cells are seeded at a seeding density of 1×10⁴-1×10⁷ cells/cm².

9. A cartilage cell culturing substrate for culturing cartilage cells which comprises a culturing surface and a plurality of projected portions formed on the culturing surface for culturing the cartilage cells in contact with top portions thereof and having an equivalent diameter and interval smaller than the equivalent diameter of the cartilage cells.

10. A cartilage cell culturing substrate according to claim 9 which further comprises a culture vessel for containing a culture solution.

11. A cartilage cell culturing substrate according to claim 10, wherein the culturing surface and the culture vessel are monolithically formed.

12. A material for regeneration of biological tissue containing cartilage cells which comprises a culturing substrate having a culturing surface on which are formed a plurality of projected portions having an equivalent diameter and interval smaller than the equivalent diameter of the cartilage cells and cartilage cells cultured in contact with top portions of a plurality of the projected portions, said cartilage cells being in the form of aggregates.

13. Cartilage cells which are cultured in contact with top portions of a plurality of projected portions formed on a culturing surface and having an equivalent diameter and an interval therebetween which are smaller than the equivalent diameter of the cartilage cells, said cartilage cells being in the form of aggregates.
